(19) United States
(12) Patent Application Publication
(10) Pub. No.: US 2014/0053838 A1
(43) Pub. Date: Feb. 27, 2014

(54) DOSE COUNTER ASSEMBLY FOR INHALER

(71) Applicants: Annaniy Berenshteyn, Ocean, NJ (US); Scott Brown, Princeton, NJ (US); Mikhail Gotliboym, Scotch Plains, NJ (US); Aleksandr Zuyev, Danville, NJ (US)

(72) Inventors: Annaniy Berenshteyn, Ocean, NJ (US); Scott Brown, Princeton, NJ (US); Mikhail Gotliboym, Scotch Plains, NJ (US); Aleksandr Zuyev, Danville, NJ (US)

(21) Appl. No.: 13/939,780

(22) Filed: Jul. 11, 2013

Related U.S. Application Data
(60) Provisional application No. 61/670,809, filed on Jul. 12, 2012.

(51) Int. Cl. A61M 15/00

(52) U.S. Cl. CPC A61M 15/0065
USPC 128/203.15

(57) ABSTRACT

Dose counter assemblies are provided herein which allow for the counting of used doses of a medical inhaler. In one aspect, one dose counter ring is utilized. This provides for dose counting but with inherent limitations in the number of doses which may be counted. In a second, and preferred, aspect of the subject invention, two cooperating does counter rings are utilized which allow for adjacent digits to be displayed, thereby allowing an increased number of doses to be counted. Advantageously, with the subject invention, due to mechanical interactions, reliable dose counting can be achieved with the subject invention.

### FIELD OF THE INVENTION

The subject invention relates to dose counters and, more particularly, to dose counters for use with medical inhalers, such as dry powder inhalers.

### BACKGROUND OF THE INVENTION

Medical inhalers, such as dry powder inhalers (DPI's) and metered dose inhalers (MDI's) are well known in the art. With such inhalers being closed containers, the amount of remaining doses of medication may be difficult to ascertain. Dose counters have been developed in the prior art useable with such inhalers to count used doses and provide an indication of remaining available doses. For example, U.S. Pat. No. 6,240,918 discloses a dose counter assembly useable with a dry powder inhaler.

### SUMMARY OF THE INVENTION

Dose counter assemblies are provided herein which allow for the counting of used doses of a medical inhaler. In one aspect, one dose counter ring is utilized. This provides for dose counting but with inherent limitations in the number of doses which may be counted. In a second, and preferred, aspect of the subject invention, two cooperating dose counter rings are utilized which allow for adjacent digits to be displayed, thereby allowing an increased number of doses to be counted. Advantageously, with the subject invention, due to mechanical interactions, reliable dose counting can be achieved with the subject invention.

These and other features of the invention will be better understood through a study of the following detailed description and accompanying drawings.

### BRIEF DESCRIPTION OF THE DRAWINGS

FIGS. 1-3 show an inhaler useable with a dose counter assembly of the subject invention; and,

FIGS. 4-18 show a dose counter assembly, and components thereof, in accordance with the subject invention.

### DETAILED DESCRIPTION OF THE INVENTION

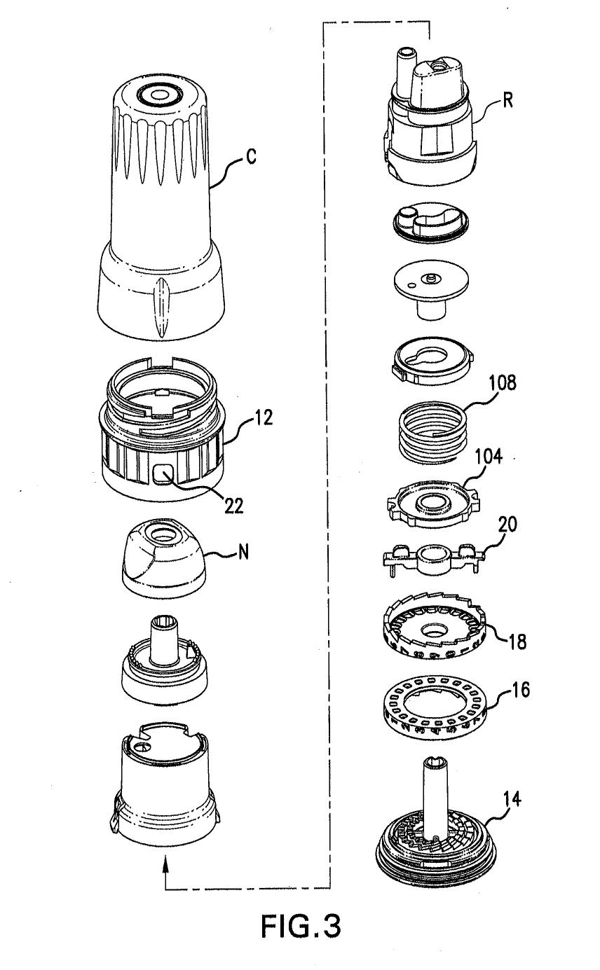

With reference to the Figures, particularly FIGS. 3-18, a dose counter assembly 10 is shown useable for counting used doses of a medical inhaler. As described below, the assembly 10 relies on rotational movement imparted thereto and, as such, is particularly well-suited f or use with dry powder inhalers. The assembly 10 can be used with the dry powder inhaler disclosed in U.S. Pat. No. 6,240,918, which is incorporated by reference herein in its entirety. As will be appreciated by those skilled in the art, metered dose inhalers utilize axial movement, typically without rotation, for use. The assembly 10 could be used with a metered dose inhaler where a portion of the axial movement is translated into rotational movement for operation of the assembly 10, as described below.

The assembly 10 may be configured in various ways to permit various number of doses to be counted. The number of doses to be counted should correlate to the capacity of the reservoir of the corresponding inhaler. In this manner, the assembly 10 may count down the proper number of doses available in the associated reservoir. The assembly 10 can be configured with a single dose counter ring, as described below, which permits counting the doses incrementally about its entire circumference. A single dose counter ring, however, is limited in the number of doses. It is preferred that the assembly 10 include two dose counter rings, as described below, which permit for two and three digit display, i.e., having at least ones and tens columns and possibly having a hundreds column.

In the preferred embodiment, the assembly 10 generally includes a housing 12, a base 14, a first dose counter ring 16, a second dose counter ring 18 and an indexer 20.

The housing 12 is generally tubular and includes a window 22 defined therein.

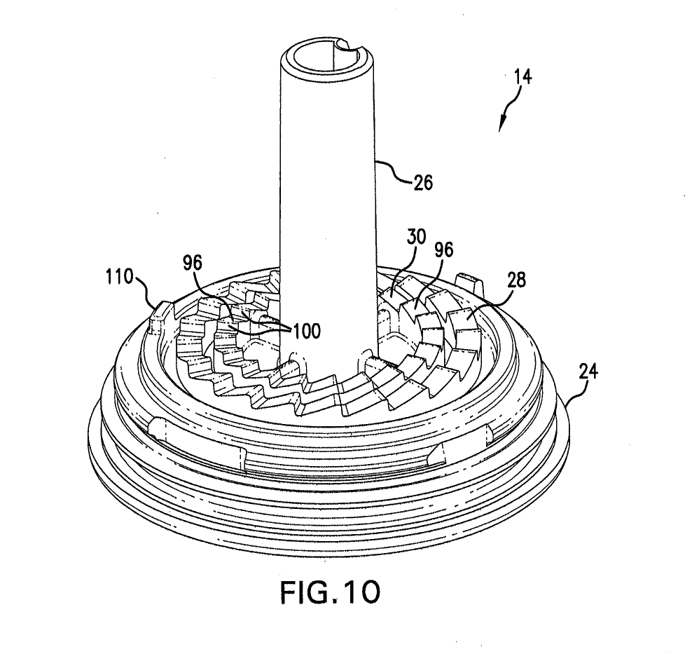

As best shown in FIG. 10, the base 14 includes a disc-shaped body 24 from which extends upwardly post 26. First and second rings of teeth 28, 30 are concentrically disposed about the post 26. Preferably, the first and second teeth 28, 30 are saw-tooth shaped. The base 14 may be fixed to the housing 12 using any known technique so as to be fixed immovably thereto. Alternatively, the base 14 may be formed integrally with the housing 12.

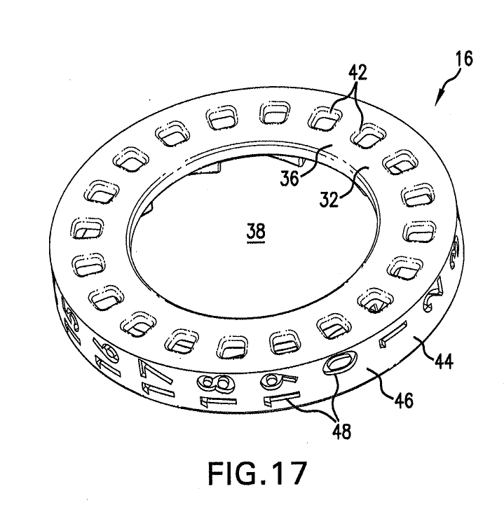

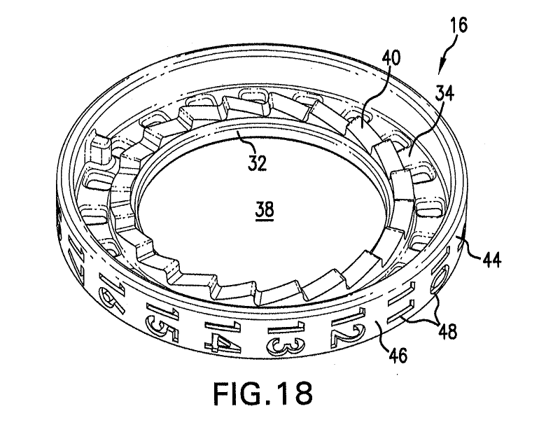

With reference to FIGS. 17 and 18, the first dose counter ring 16 includes a body 32 having opposing first and second faces 34, 36. A central aperture 38 is formed to extend through the first and second faces 34, 36 with the central aperture 38 being configured to allow passage therethrough of the post 26. As configured, the first dose counter ring 16 may be mounted onto the post 26 for rotation thereabout.

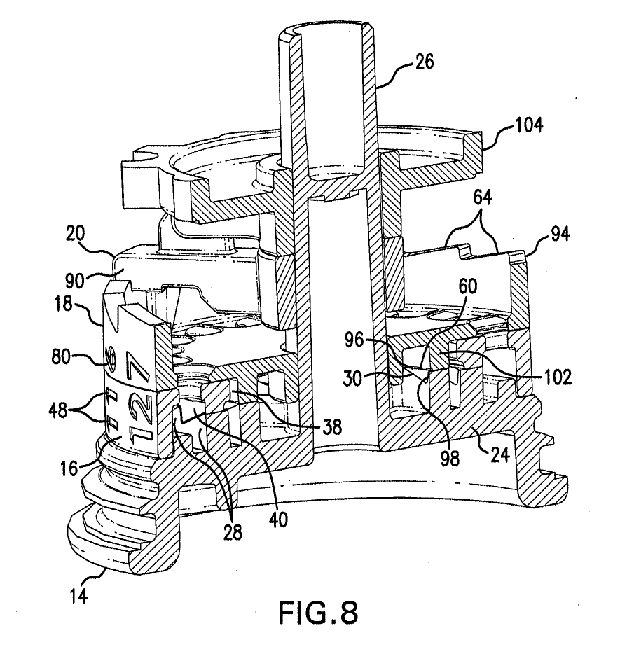

The first face 34 of the first dose counter ring 16 includes at least one ratchet tooth 40 extending therefrom formed to be nestingly received between an adjacent pair of the first teeth 28 (FIG. 8). It is preferred that a continuous ring of ratchet teeth 40 be provided formed to be simultaneously nestingly received in the first teeth 28. It is also preferred that the ratchet teeth 40 be saw-tooth shaped. At least one recess 42 is defined through the second face 36 and, preferably, extends completely through the first face 34 so as to form a through hole. A plurality of the recesses 42 may be provided, as shown in the Figures, including being disposed in a ring pattern about the central aperture 38.

Side wall 44 depends downwardly from the body 32 so as to at least partially bound the first face 34. Preferably, the side wall 44 is located at the outer periphery of the body 32. External surface 46 of the side wall 44 includes indicia 48 useable for counting doses.

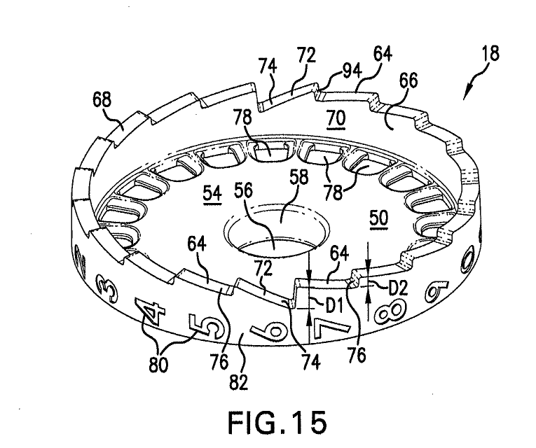

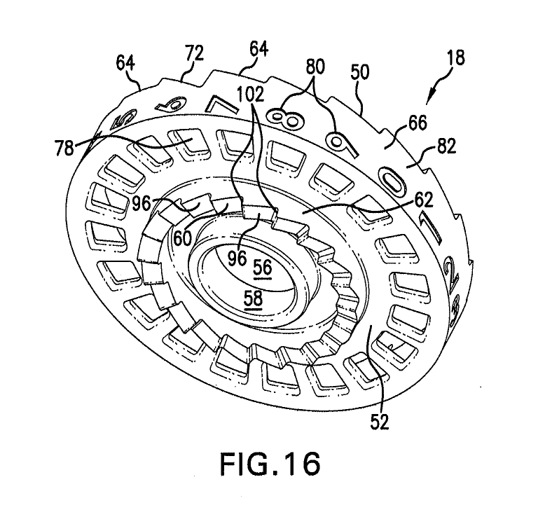

With reference to FIGS. 15 and 16, the second dose counter ring 18 includes a body 50 having opposing first and second faces 52, 54. A central aperture 56 extends between and through the first and second faces 52, 54 sized to permit mounting of the second dose counter ring 18 onto the post 26 for rotation thereabout. A collar 58 may bound all or a portion of the central aperture 56 on one or both of the first and second faces 52, 54 sized to mount onto the post 26. The collar 58 provides additional stability to the second dose counter ring 18 about the post 26.

At least one ratchet tooth 60 is formed on the first face 52. It is preferred that a plurality of ratchet teeth 60 be provided in a circular pattern so as to be simultaneously nestingly received among the second teeth 30 of the base 14 (FIG. 8). Preferably, the ratchet teeth 60 are saw-tooth shaped. As assembled, the second dose counter ring 18 lies atop the first dose counter ring 16 which, in turn, lies atop the body 24 of the base 14. To permit the ratchet teeth 60 to engage the second teeth 30 of the base 14, the central aperture 38 of the first dose counter ring 16 is provided with sufficient diameter to allow such interengagement. Preferably, the first face 52 of the second dose counter ring 18 is provided with a raised portion 62 on which the one or more ratchet teeth 60 are defined. The raised portion 62 provides sufficient extension for the one or more ratchet teeth 60 to engage the second teeth 30. Alternatively, the second teeth 30 may be raised on the body 24 so as to extend through the central aperture 38 into engagement with the one or more ratchet teeth 60. As will be appreciated by those skilled in the art, some combination of these two configurations is also permitted whereby both the second teeth 30 and the one or more ratchet teeth 60 are raised.

The second dose counter ring 18 includes a plurality of tertiary teeth 64 defined on or adjacent to the second face 54. Preferably, the tertiary teeth 64 are saw-tooth shaped. Also, preferably, the tertiary teeth 64 are provided in a continuous ring pattern. A side wall 66 may be provided to at least partially bound the second face 54. The tertiary teeth 64 may be defined on the side wall 66 either on a terminating free end 68 thereof or along an inside surface 70 thereof.

At least one of the tertiary teeth 64 is an actuator tooth 72 having a deeper recess 74 than the recesses 76 of adjacent tertiary teeth 64. In particular, with reference to FIG. 15, the actuator tooth 72 defines the deeper recess 74 having a maximum depth D1 which is greater than the maximum depth D2 of the adjacent recesses 76. Preferably, two of the actuator teeth 72 are provided which are located to be diametrically opposite on the body 50 (i.e., evenly spaced about the second dose counter ring 18).

The second dose counter ring 18 also includes at least one actuator through hole 78 formed to extend between, and through, the first and second faces 52, 54. Preferably, an actuator through hole 78 is provided adjacent to each of the actuator teeth 72. As shown in the Figures, a plurality of actuator through holes 78 may be provided spaced about the body 50, such as in a circular pattern.

Indicia 80 are provided on external surface 82 of the side wall 66 useable for counting doses.

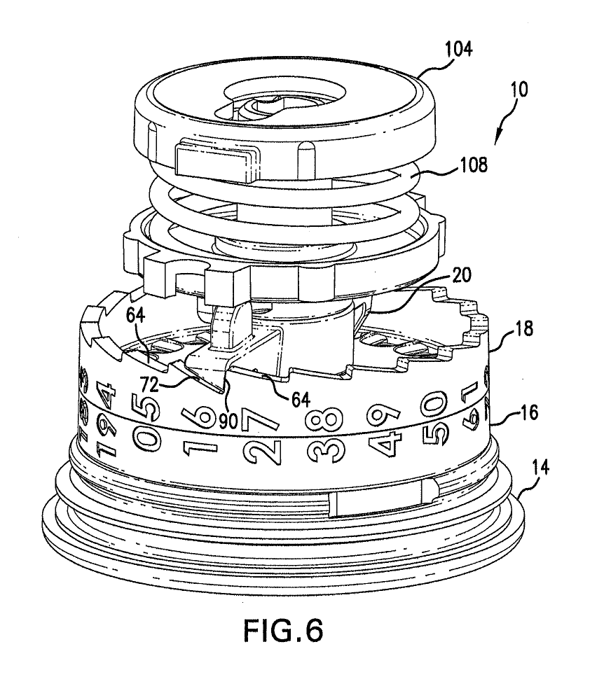

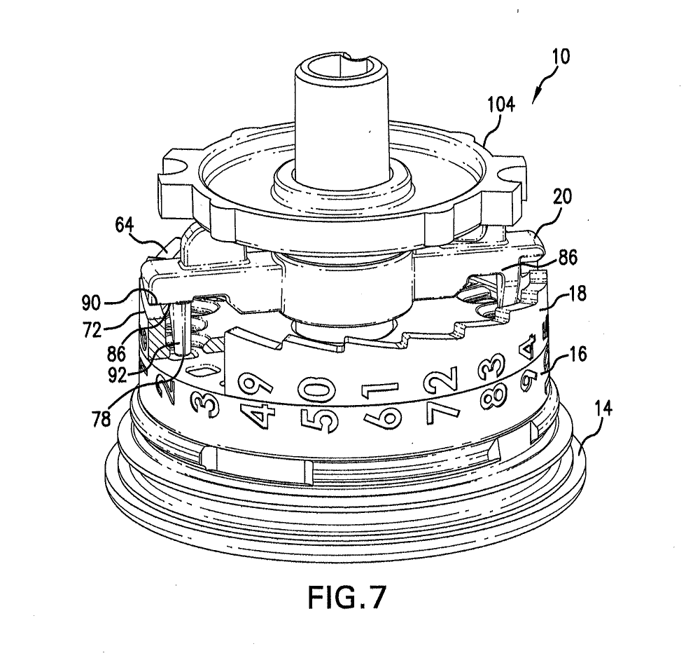

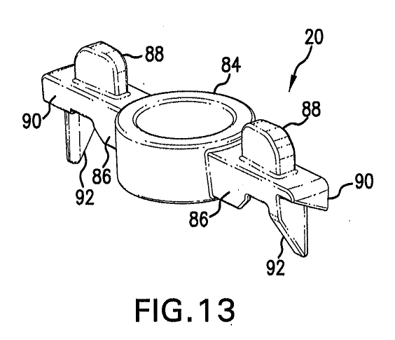

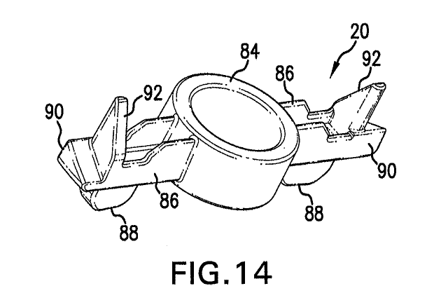

As best shown in FIGS. 13-14, the indexer 20 includes a collar 84 formed to be mounted onto the post 26 for rotation therewith. At least one arm 86, preferably two arms 86, extend from the collar 84. With more than one arm, it is preferred that the arms 86 be equiangularly located about the collar 84. For each of the arms 86, a drive tab 88 extends upwardly therefrom, with a downwardly extending engagement surface 90 and a downwardly depending secondary tab 92. The engagement surfaces 90 are located on the arms 86 to engage the tertiary teeth 64 (FIGS. 7 and 8). The actuator through holes 78 are formed to allow passage thereinto of the secondary tabs 92 (FIG. 7). With the engagement surfaces 90 being nestingly received within the deeper recesses 74 of the actuator teeth 72 (FIG. 6), the secondary tabs 92 are provided with sufficient length to pass through the actuator through holes 78 into engagement with the recesses 42 of the first dose counter ring 16.

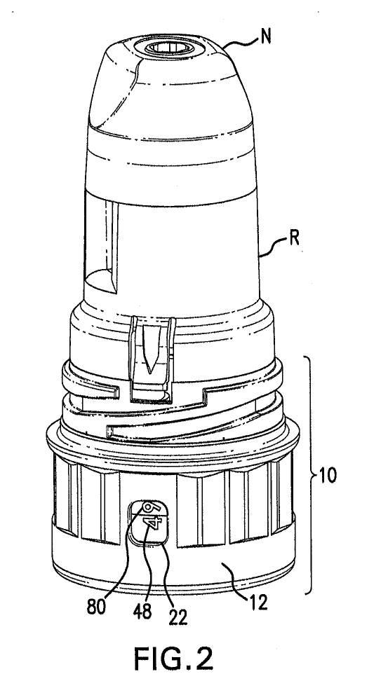

With reference to the Figures, the assembly 10 is formed by stacking in order, within the housing 12, the first dose counter ring 16, the second dose counter ring 18, and the indexer 20 on the post 26. In this state, simultaneously, the ratchet teeth 40 are in engagement with the first teeth 28 and the ratchet teeth 60 are in engagement with the second teeth 30, as shown in FIG. 8. Also, the indicia 48 and the indicia 80 are aligned so as to be viewable together. The assembly 10 is configured so that one set of indicia 48, 80 is viewable at a given instance through the window 22 (FIG. 2). This provides the dose count. With adjustment of the assembly 10, the indicia 48, 80 are adjusted to reflect an updated count.

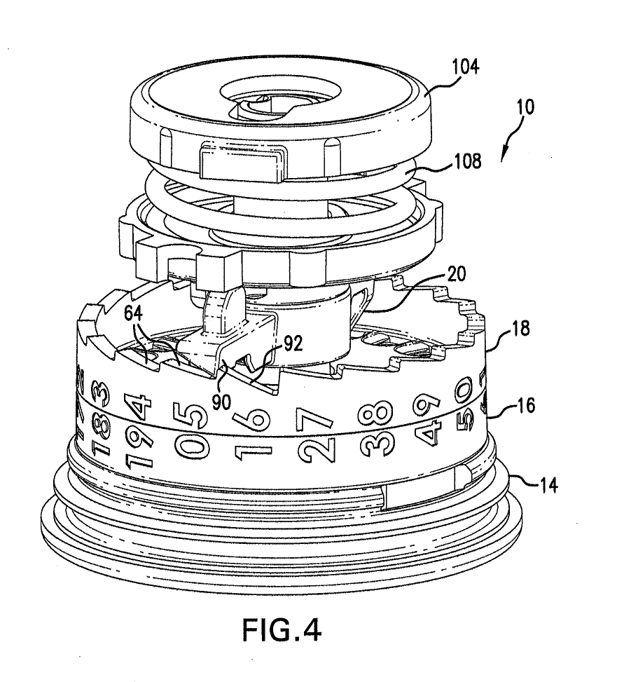

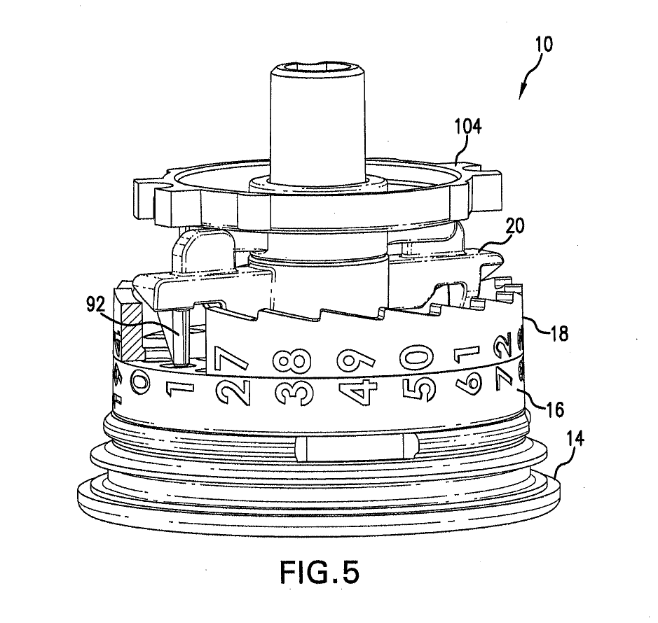

In a rest position, the engagement surfaces 90 of the indexer 20 are in engagement with the tertiary teeth 64 (FIG. 4). With the engagement surfaces 90 engaging the tertiary teeth 64 and not the actuator teeth 72, the actuator through holes 78 are preferably positioned to be spaced below so as to be out of contact with the secondary tabs 92 in a rest state and/or configured and positioned to allow passage thereinto of the secondary tabs 92 in the rest state to avoid contact between the secondary tabs 92 and the second dose counter ring 18 (FIG. 5).

In operation, rotational force is applied to the drive tabs 88 in a direction with the engagement surfaces 90 pressing against vertical sides 94 of the tertiary teeth 64. This results in rotational force being applied to the secondary dose counter ring 18. The second teeth 30 and the ratchet teeth 60 are configured so that in a rest state, angled faces 96 of the second teeth 30 are in engagement with angled faces 98 of the ratchet teeth 60. With rotational movement imparted to the second dose counter ring 18, the angled faces 98 are caused to slide along the angled faces 96 in a direction away from vertical faces 100 of the second teeth 30. This causes the second dose counter ring 18 to rise relative to the base 14. With sufficient radial displacement, each of the ratchet teeth 60 by-passes an individual of the second teeth 30. The ratchet teeth 60 then are nestingly received by the next adjacent pair of the second teeth 30. This incremental adjustment corresponds to individual adjustment of one of the indicia 80 on the second dose counter ring 18. To re-set the assembly 10 for a further dose counting, the indexer 20 is caused to be rotated in a reverse direction, with the engagement surfaces 90 being caused to by-pass individual tertiary teeth 64. During this reverse motion, vertical faces 102 of the ratchet teeth 60 engage against the vertical faces 100 of the second teeth 30 thereby preventing reverse rotation of the second dose counter ring 18.

As will be appreciated by those skilled in the art, incremental dose counting can be achieved by the assembly 10 by rotation and re-setting of the indexer 20 as described above. With sufficient number of adjustments of the second dose counter ring 18, the first dose counter ring 16 may be adjusted, such as for example to reflect a change in the tens or one hundreds column. By way of example, the first dose counter ring 16 may in an initial state show the indicia 48 as the number 1 through the window 22 with the indicia 80 on the second dose counter ring 18 being shown incrementally as digits between 9 and 0. Where the indicia 48 and the indicia 80 collectively show the number “10”, upon the next counting of a dose, the first dose counter ring 16 may be adjusted to show the digit “0” and with the indicia 48 and 80 collectively showing “09”.

To permit adjustment of the first dose counter ring 16, the actuator teeth 72 are located to permit adjustment of the first dose counter ring 16 upon a certain number of adjustments of the second dose counter ring 18. For example, the actuator teeth 72 may be located to permit an adjustment of the first dose counter ring 16 on every tenth adjustment of the second dose counter ring 18. With the engagement surfaces 90 engaging the deeper recesses 74 of the actuator teeth 72 (FIG. 6), the secondary tabs 92 are caused to pass through the actuator through holes 78 (FIG. 7) and into engagement with the recesses 42 of the first dose counter ring 16. With rotation of the indexer 20 as described above, rotational forces are imparted to the first dose counter ring 16 due to the interengagement of the secondary tabs 92 and the recesses 42 resulting in the ratchet teeth 40 by-passing an individual of the first teeth 28, in the same manner described above with respect to the second teeth 30 and the ratchet teeth 60. Reverse rotation of the indexer 20 causes the secondary tabs 92 to separate from the recesses 42. Due to the interengagement of the first teeth 28 and the ratchet teeth 40, inadvertent rotation of the first dose counter ring 16 is avoided.

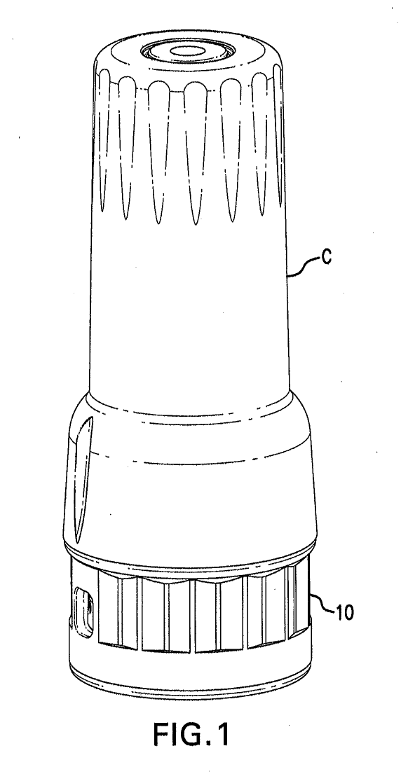

As will be appreciated by those skilled in the art, rotational force may be imparted to the drive tabs 88 of the indexer 20 in various manners. By way of non-limiting example, and in a manner like that disclosed in U.S. Pat. No. 6,240,918, a spring retainer 104 may be provided adjacent to the indexer 20 (FIG. 4). The spring retainer 104 is fixed to a portion of an inhaler, as known in the art, which is rotatable relative to the housing 12. In this manner, the spring retainer 104 may be rotated relative to the assembly 10. For example, with reference to FIGS. 1 and 2, reservoir R is coupled to the spring retainer 104 so as to be rotatable therewith. Rotational force may be imparted to the reservoir R by mounting and dismounting closure cap C, in a manner as disclosed in U.S. Pat. No. 6,240,918. Any arrangement for allowing delivery of medicament from the reservoir R and through nozzle N may be utilized.

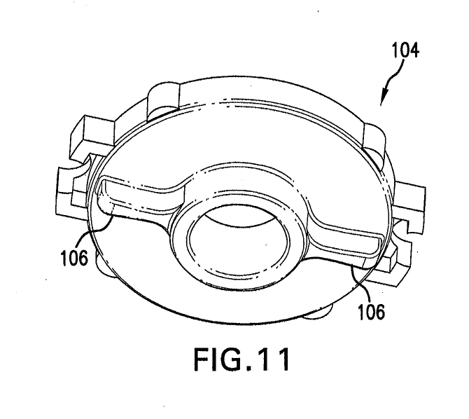

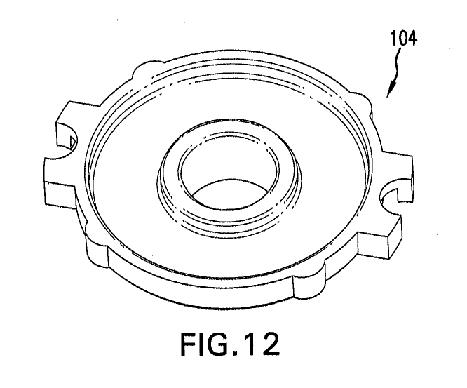

As best shown in FIGS. 11 and 12, the spring retainer 104 includes one or more driving ribs 106 configured and located to engage the drive tabs 88 upon rotation of the spring retainer 104 relative to the indexer 20. As will be appreciated by those skilled in the art, depending on the operation of the associated inhaler, the spring retainer 104 may engage the indexer 24 over a limited range of its motion.

To ensure that the components of the assembly 10 remain in tight engagement, a spring, or other biasing means, 108 is provided to apply biasing force against the assembly 10 in a direction of the base 14. Preferably, the spring 108 is located to press against the spring retainer 104. With the first and second dose counter rings 16, 18 rising during displacement, as described above, this motion may be resiliently absorbed by the spring 108.

As will be appreciated by those skilled in the art, the first dose counter ring 16 may be used alone, without the second dose counter ring 18, wherein the indexer 20 is caused to urge the first dose counter ring 16 incrementally about the base 14. With this arrangement, the tertiary teeth 64 will be provided on the first dose counter ring 16. The actuating teeth 72 and the actuator through holes 78 are not necessary.

The indicia 48, 80 are preferably arranged to count down the available number of doses so that showing of zero indicates no remaining doses. Colors or other indicia may be used in combination with numbers, or alternatively alone, to indicate the level of remaining doses. For example, colors, such as green, yellow, and red, may be provided to cover ranges of doses with red, for example, providing an alert of low supply.

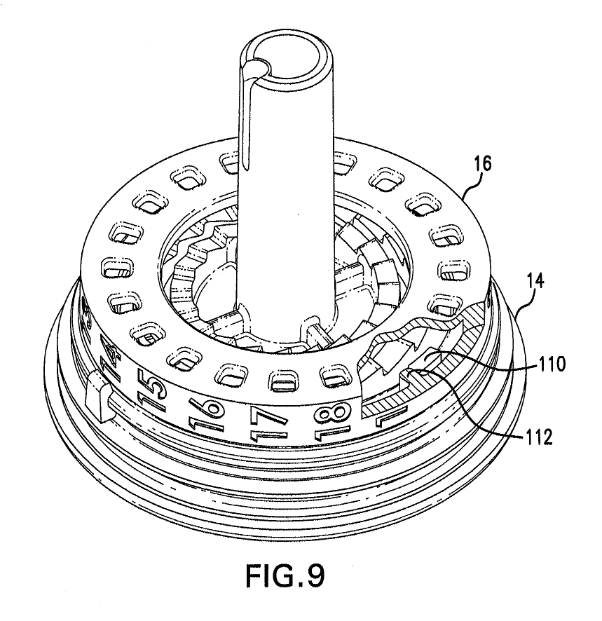

A stop 110 may be provided which is engaged by one or both of the first dose counter ring 16 and the second dose counter ring 18 upon a predetermined number of adjustments of the assembly 10 corresponding to a predetermined number of administered doses. For example, as shown in FIG. 9, the stop 110 may be located on the base 14 so as to be interferingly engaged by a stop member 112 formed on the first dose counter ring 16 upon a predetermined number of adjustments of the first dose counter ring 16. This allows for stopping further adjustment, and, thus, stopping further possible dosing. A certain number of extra doses may be provided for the inhaler, beyond the rated amount, to best ensure that the inhaler can provide all doses to be counted. The use of the stop 110 prevents dosing of any residual medicament left beyond the fully counted number of doses.

What is claimed is:

1. A dose counter assembly for counting used doses of an inhaler, said dose counter comprising:
a housing having a window defined therein;
a base having a plurality of saw-tooth shaped teeth disposed thereon in a circular pattern;
a dose counter ring having at least one ratchet tooth extending therefrom formed to be nestingly received between an adjacent pair of said teeth on said base, said dose counter ring including indicia on external portions thereof; and,
an indexer formed to rotate said dose counter ring by individual increments about said base with said ratchet teeth by-passing said teeth of said base,
wherein said dose counter ring being disposed in said housing such that said indicia is viewable through said window as said dose counter ring is caused to rotate, said indicia providing an indication of the number of used doses of the inhaler.

2. A dose counter assembly for counting used doses of an inhaler, said dose counter comprising:
a housing having a window defined therein;
a base having concentric first and second rings of saw-tooth shaped teeth disposed thereon;
a first dose counter ring having a disc-shaped body with opposing first and second surfaces, said first surface having at least one ratchet tooth formed to be nestingly received between an adjacent pair of said first teeth on said base, at least one recess defined through said second face, a central aperture extending between, and through, said first and second faces, and first indicia on external portions of said first dose counter ring;
a second dose counter ring having a disc-shaped body with opposing first and second faces, a portion of said first face being raised and configured to at least partially pass through said central aperture of said first dose counter ring, at least one secondary ratchet tooth extending from said raised portion formed to be nestingly received between an adjacent pair of said second teeth on said base, a plurality of tertiary saw-tooth shaped teeth formed on said second face, at least one of said tertiary teeth being an actuator tooth defining a deeper recess than adjacent said tertiary teeth, an actuator through hole being defined between, and through, said first and second faces, and second indicia on external portions of said second dose counter ring;
an indexer formed with an engagement surface formed to be nestingly received between an adjacent pair of said tertiary teeth, and at least one protruding tab having sufficient length to extend through said actuator through hole into engagement with said recess of said first dose counter ring with said engagement surface of said indexer being nestingly received within said deeper recess defined by said actuator tooth,
wherein said indexer causes said second dose counter ring to rotate due to interengagement between said engagement surface and at least one of said tertiary teeth,
wherein said indexer causes said first dose counter ring to rotate due to interengagement between said tab and said recess of said first dose counter ring, and,
wherein said first and second dose counter rings are disposed in said housing such that said first and second indicia are viewable through said window as said first and second dose counter rings are caused to rotate, said first and second indicia providing an indication of the number of used doses of the inhaler.
